SEQUENCE LISTING (26 sequences)

SEQ ID NO: 1 - DNA, 681 bp, Corynebacterium diphtheriae
atgaaagatttggtcgataccacagaaatgtatctgcggaccatctacgagctggaagaagagggagtaactccccttcgcgcacgcatcgccgaacgcctcgatcagtcaggccctacagtcagccaaacagttgcccgcatggaacgtgacgggctcgttgtagttgcgtctgaccgtagtcttcaaatgacgcccactgggcgcgctttagccaccgccgtaatgcgtaaacatcgcctcgcagagcgcctccttacagacattattggcttagatatccacaaggtgcacgatgaagcatgccgctgggagcacgtcatgagcgacgaagtagagcggcggcttgttgatgtcctcgaggacgtcacccgctccccctttggcaacccaatcccaggtctcgatgaacttggcgtctccataaaaaagaaggaaggaccgggcaaacgtgccgtggatgtagcccgtgccacccccagagacgtaaagattgttcaaatcaacgagatattgcaagtagattctgaccagtttcaggctctgatcgacgcaggcattagaattggaacgaccgtcacgctcagcgatgtagacggtcgcgtgattattacgcacggtgaaaaaacagtagaacttatcgacgacctagctcacgcagtacgaatcgaagaaatctaa

SEQ ID NO: 2 - protein, 226 aa, Corynebacterium diphtheriae
MKDLVDTTEMYLRTIYELEEEGVTPLRARIAERLEQSGPTVSQTVARMERDGLVVVASDRSLQMTPTGRTLATAVMRKHRLAERLLTDIIGLDINKVHDEACRWEHVMSDEVERRLVKVLKDVSRSPFGNPIPGLDELGVGNSDAAAPGTRVIDAATSMPRKVRIVQINEIFQVETDQFTQLLDADIRVGSEVEIVDRDGHITLSHNGKDVELLDDLAHTIRIEEL

SEQ ID NO: 3 - DNA, 660 bp, Streptococcus suis
atgacaccaaacaaagaagattacctaaaatgtatttatgaactgggtcaattagaccaaaaaattaccaataaactcatcgcagagaagatggccttctccgcaccagccgtttccgaaatgctcaaaaaaatggtagccgaagagctcatttctaaggatgccaaggcaggttatctcctcagtcaaactgcccttgaaatggtagccagcctctatcgcaaacaccgcttgattgaggtattcttagttgagcaacttggctactctccagaagaagtacatgaagaggctgagattttagaacacaccgtttcagatcactttatcaatcgcctagacctgctactggaacagcctcaaacttgtcctcacgggggaagcattcctcaagcaggacaaccgctcatcgaacgctaccagacacggctgtcacagctaactgagacagggaactaccagcttgtccgtatccatgacttctatcaactccttcagtacttggaacaacatgaattagctgtcggtgatttactaaccgtcacagccttcgaccagtttgcccagaccatcaccatccagtacaaggacaaagagctcgccgtcccaacagccatcgctcaacaattattcatcgaaaaaagcaatcgcccagcctaa

SEQ ID NO: 4 - protein, 219 aa, Streptococcus suis
MTPNKEDYLKCIYELGQLDQKITNKLIAEKMAFSAPAVSEMLKKMVAEELISKDAKAGYLLSQTALEMVASLYRKHRLIEVFLVEQLGYSPEEVHEEAEILEHTVSDHFINRLDLLLEQPQTCPHGGSIPQAGQPLIERYQTRLSQLTETGNYQLVRIHDFYQLLQYLEQHELAVGDLLTVTAFDQFAQTITIQYKDKELAVPTAIAQQLFIEKSNRPA

SEQ ID NO: 5 - DNA, 29 bp, Artificial
  Primer
gggaattcgctacagctacagctgacttg

SEQ ID NO: 6 - DNA, 32 bp, Artificial
  Primer
cgctcagcttgtttacatgagaactcgctttc

SEQ ID NO: 7 - DNA, 32 bp, Artificial
  Primer
gaaagcgagttctcatgtaaacaagctgagcg

SEQ ID NO: 8 - DNA, 28 bp, Artificial
  Primer
gggaattcgacgaatgacggatactatc

SEQ ID NO: 9 - DNA, 21 bp, Artificial
  Primer
ccagtgagcgcgcgtaatacg

SEQ ID NO: 10 - DNA, 21 bp, Artificial
  Primer
ggtatactactgacagcttcc

SEQ ID NO: 11 - DNA, 16 bp, Artificial
  Primer
cacagccactcttggc

SEQ ID NO: 12 - DNA, 18 bp, Artificial
  Primer
gtcttgcagcctttaacc

SEQ ID NO: 13 - DNA, 19 bp, Artificial
  Primer
gaactgggtcaattagacc

SEQ ID NO: 14 - DNA, 19 bp, Artificial
  Primer
gagctctttgtccttgtac

SEQ ID NO: 15 - DNA, 20 bp, Artificial
  Primer
tggaaataagacttagaagc

SEQ ID NO: 16 - DNA, 20 bp, Artificial
  Primer
cgactcatagaattatttcc

SEQ ID NO: 17 - DNA, 40 bp, Artificial
  Primer
cggaattcctttcaccttctaggtaaatcacatcaatacc

SEQ ID NO: 18 - DNA, 39 bp, Artificial
  Primer
gcaccctgcggtcttatcctttacaatccagccttgtgc

SEQ ID NO: 19 - DNA, 39 bp, Artificial
  Primer
gataagaccgcagggtgcatgatcactttgagcttatcc

SEQ ID NO: 20 - DNA, 41 bp, Artificial
  Primer
cggaattcgtgatgttgttgttgctgatcgcttggtgtatc

SEQ ID NO: 21 - DNA, 25 bp, Artificial
  Primer
gcagagagaatgaaggtttctgcac

SEQ ID NO: 22 - DNA, 26 bp, Artificial
  Primer
caattccttatctgcataagtgatgg

SEQ ID NO: 23 - DNA, 25 bp, Artificial
  Primer
ctattatctaacagagcaagggcag

SEQ ID NO: 24 - DNA, 24 bp, Artificial
  Primer
tgttttgttgatttcgattagtgg

SEQ ID NO: 25 - DNA, 20 bp, Artificial
  Primer
tggaaataagacttagaagc

SEQ ID NO: 26 - DNA, 20 bp, Artificial
  Primer
cgactcatagaattatttcc

(19) United States
(12) Patent Application Publication
(10) Pub. No.: US 2015/0140032 A1
(43) Pub. Date: May 21, 2015

(54) VACCINE

(71) Applicant: MOREDUN RESEARCH INSTITUTE, Midlothian (GB)

(72) Inventor: Michael Fontaine, Edinburgh (GB)

(21) Appl. No.: 14/402,931

(22) PCT Filed: May 22, 2013
(86) PCT No.: PCT/GB2013/051337

§ 371 Date: Nov. 21, 2014

(30) Foreign Application Priority Data
May 22, 2012 (GB) 1209014.8

(51) Int. Cl. A61K 39/09; A61K 45/06

(52) U.S. Cl. CPC A61K 39/092; A61K 45/06; A61K 2039/521
USPC 424/200.1; 435/252.3

(57) ABSTRACT

The present invention is based on the finding that microorganisms can be modified so as to express certain factors important in generating or raising host immune responses. In particular, the invention provides modified microorganisms which, when subjected to conditions which would be expected to suppress or reduce the expression, function and/or activity of certain factors, exhibit increased (often significantly increased) expression, function and/or activity of those factors. The invention provides a modified microorganism capable of expressing at least one factor under conditions in which a wild-type (or unmodified) strain of the same microorganism, exhibits inhibited expression of the at least one factor.

### FIELD OF THE INVENTION

The present invention provides modified microorganisms for raising host immune responses as well as vaccines and vaccine compositions comprising the same. In particular, the invention provides a modified Streptococcus, which may form the basis of an improved vaccine for treating and/or preventing diseases.

### BACKGROUND OF THE INVENTION

Several species of the genus Streptococcus are the causative agents of a number of diseases in humans and animals. In humans, the most frequently-encountered pathogenic species is S. pneumoniae (the pneumococcus), which causes sinusitis and otitis media, but also life-threatening conditions including pneumonia, sepsis, osteomyelitis, endocarditis, septic arthritis and meningitis among others. Second most frequently encountered in humans is the Group A Streptococcus (GAS), S. pyogenes, which is responsible for pharyngitis, glomerulonephritis, acute rheumatic fever, scarlet fever and on occasion, necrotising fasciitis. Other species, such as S. mutans, may constitute part of the normal human microflora, yet may pose a disease risk under the right conditions.

Animal diseases caused by streptococci are no-less significant than those in humans. For example, S. suis causes respiratory disease, joint infections, skin conditions and meningitis in pigs. Furthermore, this organism is zoonotic, and may be acquired occupationally, resulting in meningitis, endocarditis and/or septicaemia. Another significant animal pathogen is S. equi, which causes strangles in horses. In the dairy industry, one of the major causes of mastitis in lactating cattle is S. uberis, while S. dysgalactiae subsp. dysgalactiae also contributes to the incidence of this disease. Likewise, S. agalactiae is also recognised as a cause of mastitis, but is also responsible for causing a range of other diseases in a diverse number of species including fish, aquatic mammals and humans.

While vaccines against some of the major Streptococci pathogens exist, many are unreliable, inducing weak, short-lived and/or ineffective immune responses. As such, there is a requirement for new vaccines against streptococci which induce immunity in human and animal hosts.

### SUMMARY OF THE INVENTION

The present invention is based on the finding that microorganisms can be modified so as to express certain factors important in generating or raising host immune responses. In particular, the invention provides modified microorganisms which, when subjected to conditions which would be expected to suppress or reduce the expression, function and/or activity of certain factors, exhibit increased (often significantly increased) expression, function and/or activity of those factors. In one embodiment, the factors may be virulence factors.

The modified microorganisms provided by this invention may find application as agents for generating or raising immune responses and as vaccines or vaccine compositions to protect against a variety of diseases and/or conditions and/or to prevent or reduce host colonisation/infection by one or more pathogens.

In a first aspect, the present invention provides a modified microorganism capable of expressing at least one factor under conditions in which a wild-type (or unmodified) strain of the same microorganism, exhibits inhibited expression of the at least one factor.

It should be understood that while this invention may be described as “comprising” one or more features, the term “comprising” encompasses aspects and embodiments which “consist essentially of” or “consist of” the noted feature(s).

As such, the invention may provided a modified bacterium capable of expressing at least one factor under conditions in which a wild-type (or un-modified) strain of the same bacterium, exhibits inhibited expression of the at least one factor.

The modified bacterium may be a modified Streptococcus species wherein, under environmental conditions suppressing or inhibiting the expression of a factor or factors in a wild-type or un-modified form of the same Streptococcus species, the modified Streptococcus species express the factor or factors. It should be understood that references to “Streptococcus species” encompass not only the specific species, S. suis and S. equi, but other species such as, for example, S. pyrogenes, S. epidermidis, S. pneumoniae, S. gordonii and/or S. mutans.

The modified Streptococcus species may be a modified Streptococcus suis or a modified Streptococcus equi wherein, under environmental conditions suppressing or inhibiting the expression of a factor or factors in a wild-type or un-modified S. suis or S. equi, the modified S. suis and S. equi express the factor or factors.

The term “factors” should be understood as encompassing proteinaceous compounds (for example proteins, peptides, amino acids and/or glycoproteins as well as small organic compounds, lipids, nucleic acids and/or carbohydrates produced by microorganisms. Many of these factors are expressed internally—i.e. within the cytoplasm of the microorganism; such factors may be classed as “internal” or “cytoplasmic”. The term “factors” may also encompass microbial factors which are secreted from the cell and/or targeted to the microbial cell wall as membrane-bound or transmembrane factors. The term “factors” may further comprise antigenic or immunogenic compounds which elicit or generate host immune responses. Such factors may include those collectively known as “virulence determinants/factors” and/or “pathogenicity factors”. One of skill will appreciate that microbial factors which are also virulence determinants/factors and/or pathogenicity factors, may comprise, for example, those which facilitate microbial attachment to host surfaces or cells and/or host cell invasion as well as those involved in toxin production and/or the toxins themselves. In view of the above, the term “factors” as used herein may comprise microbial cell wall, membrane and/or transmembrane structures such as proteins or compounds which mediate or facilitate host adherence or colonisation, pili and/or secreted enzymes, compounds and/or toxins. The term “factors” may further comprise compounds involved in metal ion acquisition.

One of skill will appreciate that in wild-type microorganisms, for example wild-type bacteria including Streptococcus species (such as, for example, S. suis and/or S. equi), the expression, function and/or activity of one or more factor(s) may be directly or indirectly regulated by one or more exogenous and/or endogenous elements.

An endogenous element may directly or indirectly regulate the activity, expression and/or function of a microbial factor. An “endogenous” regulatory element may be a microbial element which regulates the function, expression and/or activity of one or more microbial factors. In contrast, an “exogenous” regulatory element may comprise an element which is not produced by, or is not a product of, a microorganism, but which directly or indirectly regulates the expression, function and/or activity of a factor expressed by that microorganism.

One of skill will appreciate that in some cases, exogenous and/or endogenous regulatory elements of the type described herein, act as global regulatory elements. Global regulatory elements may regulate and/or control the expression, function and/or activity of a plurality of microbial factors.

The exogenous regulatory element may comprise an environmental element. One of skill will appreciate that an environmental regulatory element may comprise a particular nutrient, compound, vitamin, metabolite, mineral, ion, electrolyte and/or salt. Additionally, or alternatively an environmental regulatory element may take the form of a physical condition such as, for example, a particular temperature, gas ratio, osmolairty and/or pH.

One of skill will readily understand that the presence and/or absence of one or more (exogenous) environmental regulatory elements may directly modulate the expression, function and/or activity of one or more microbial factor(s). In other cases, the presence and/or absence of one or more environmental regulatory element(s) may modulate the expression, function and/or activity of one or more endogenous microbial regulatory element(s) (for example an endogenous (microbial) global regulatory element) which in turn effects the expression, function and/or activity of one or more microbial factor(s).

Modified microorganisms provided by this invention may lack one or more endogenous regulatory/control elements. In one embodiment, the modified microorganisms may lack one or more environmentally-sensitive or responsive regulatory/control elements. As a consequence of these modifications, the modified microorganisms described herein are characterised by the expression/function and/or activity of one or more factors in environments (or under conditions) which would normally (i.e. in a wild-type or unmodified strain) suppress or inhibit the expression, function and/or activity of said factors.

The factors expressed by the modified microorganisms described herein may comprise factors, the expression, function and/or activity of which is normally associated with, controlled/regulated by, dependent on and/or sensitive to, the presence and/or absence of metal ions such as, for example iron (Fe²⁺) and/or manganese (Mn²⁺).

Advantageously, and where the invention relates to, for example, modified Streptococcus, such factors may comprise one or more Streptococcus antigens/immunogens (virulence factors) said antigens and/or immunogenes being capable of generating, raising and/or eliciting a host immune response.

Accordingly, the invention may relate to a modified species of the Streptococcus genus, expressing at least 1 factor under conditions comprising manganese and/or iron concentrations which inhibit the expression of said factor in wild-type or unmodified strains of the same organism.

The modified microorganisms provided by this invention may comprise one or more genetic modification(s) which directly and/or indirectly affect the expression, activity and/or function of one or more microbial regulatory elements (including global regulatory elements). A genetic modification which affects the expression, function and/or activity of a microbial regulatory element, may comprise one or more mutations in the sequence of a gene encoding said regulatory element. In contrast, a genetic modification which indirectly affects the expression, function and/or activity of a microbial regulatory element, may comprise one or more mutations in the sequence of a gene or genes which encode other elements or factors which themselves affect the activity, function and/or expression of the regulatory element.

A genetic modification may comprise one or more alterations in a nucleic acid sequence. For example, a nucleic acid sequence may be modified by the addition, deletion, inversion and/or substitution of one or more nucleotides of a sequence. One of skill will appreciate that a genetic modification may effect the expression, function and/or activity of the nucleic acid sequence harbouring the modification and/or the expression, function and/or activity of the protein or peptide encoded thereby.

Advantageously, modified microorganisms provided by this invention comprise genetic lesions resulting in the (“in-frame”) deletion of nucleic acid sequences. Furthermore, the modified microorganisms of this invention may lack exogenous nucleic acid—for example nucleic acids derived from vectors (for example plasmids and the like). As such, when compared to isogenic, wild-type parent strains, a modified microorganism (for example a modified Streptococcus) of this invention may be identical except for the mutation or deletion of sequences encoding one or more regulatory elements.

In Corynebacterium diphtheriae, a number of virulence factors (including diphtheria toxin (encoded by the tox gene)) are regulated by the metal ion-activated global regulatory element, DtxR (product of the dtxR gene). Other bacterial species including, for example other Corynebacterium and Streptococcus species, comprise global regulators which are structurally and/or functionally homologous (and/or (substantially) identical) to the dtxR/DtxR gene/protein of C. diphtheriae.

Without wishing to be bound by theory, the inventors have discovered that microorganisms (for example species belonging to the Streptococcus genus) exhibiting modified expression, function and/or activity of a gene and/or protein homologous to the dtxR gene and/or DtxR protein of Corynebacterium diphtheriae, represent exemplary vaccine candidates.

In view of the above, this invention may provide modified microorganisms capable of expressing at least one factor under conditions in which a wild-type (or un-modified) strain of the same microorganism, exhibits inhibited expression of the at least one factor, wherein the modified microorganism lacks (i) a functional dtxR homologue, (ii) a gene functionally equivalent to dtxR and/or (iii) a gene or protein which is “dtxR like”. For convenience, options (i), (ii) and (iii) above will, hereinafter, be collectively referred to as “dtxR homologues”. It should be understood that dtxR homologues encompassed by this invention (including genes/proteins which are dtxR-like) may exhibit variable (perhaps low) sequence homology/identity with the dtxR gene/protein of Corynebacterium diphtheriae but a high degree of functional homology/identity with dtxR—in other words, the dtxR homologues described herein are metalo-regulators which, through binding metal ions, exert an effect on gene expression.

It should be understood any gene and/or protein being described as “functionally homologous” to the dtxR and/or DtxR gene/protein of C. diphtheriae, is a gene and/or protein which exhibits metalo-regulator activity characteristic of, or similar to the metalo-regulator activity of the dtxR/DtxR gene/protein of C. diphtheriae.

The sequence encoding the C. diphtheriae dtxR gene is provided as SEQ ID No:1, below.

| SEQ ID NO: 1 | |
|---|---|
| 1 | atgaaagatt tggtcgatac cacagaaatg tatctgcgga ccatctacga gctggaagaa |
| 61 | gagggagtaa ctccccttcg cgcacgcatc gccgaacgcc tcgatcagtc aggccctaca |
| 121 | gtcagccaaa cagttgcccg catggaacgt gacgggctcg ttgtagttgc gtctgaccgt |
| 181 | agtcttcaaa tgacgcccac tgggcgcgct ttagccaccg ccgtaatgcg taaacatcgc |
| 241 | ctcgcagagc gcctccttac agacattatt ggcttagata tccacaaggt gcacgatgaa |
| 301 | gcatgccgct gggagcacgt catgagcgac gaagtagagc ggcggcttgt tgatgtcctc |
| 361 | gaggacgtca cccgctcccc ctttggcaac ccaatcccag gtctcgatga acttggcgtc |
| 421 | tccataaaaa agaaggaagg accgggcaaa cgtgccgtgg atgtagcccg tgccaccccc |
| 481 | agagacgtaa agattgttca aatcaacgag atattgcaag tagattctga ccagtttcag |
| 541 | gctctgatcg acgcaggcat tagaattgga acgaccgtca cgctcagcga tgtagacggt |
| 601 | cgcgtgatta ttacgcacgg tgaaaaaaca gtagaactta tcgacgacct agctcacgca |
| 661 | gtacgaatcg aagaaatcta a |

An exemplary DtxR protein sequence has been deposited as accession No: YP_{—}005162868. A sequence of the dtxR protein is given as SEQ ID NO: 2 below:

| SEQ ID NO: 2 | |
|---|---|
| 1 | mkdlvdttem ylrtiyelee egvtplrari aerleqsgpt vsqtvarmer dglvvvasdr |
| 61 | slqmtptgrt latavmrkhr laerlltdii gldinkvhde acrwehvmsd everrlvkvl |
| 121 | kdvsrspfgn pipgldelgv gnsdaaapgt rvidaatsmp rkvrivqine ifqvetdqft |
| 181 | qlldadirvg seveivdrdg hitlshngkd vellddlaht irieel |

Homologous and/or identical dtxR and/or DtxR genes/proteins may encompass those encoded by nucleic acid and/or amino acid sequences which exhibit at least about 30%, 40%, 50%, 60%, 65%, 70%, 75%, 80%, 85%, 90%, 91%, 92%, 93%, 94%, 95%, 96%, 97%, 98% or 99% homology or identity with SEQ ID NOS: 1 or 2 above or fragments thereof.

The degree of (or percentage) “homology” between two or more (amino acid or nucleic acid) sequences may be determined by aligning the sequences and determining the number of aligned residues which are identical and adding this to the number of residues which are not identical but which differ by redundant nucleotide substitutions—the redundant nucleotide substitution having no effect upon the amino acid encoded by a particular codon, or conservative amino acid substitutions. The combined total is then divided by the total number of residues compared and the resulting figure is multiplied by 100—this yields the percentage homology between aligned sequences.

A degree of (or percentage) “identity” between two or more (amino acid or nucleic acid) sequences may also be determined by aligning the sequences and ascertaining the number of exact residue matches between the aligned sequences and dividing this number by the number of total residues compared—multiplying the resultant figure by 100 would yield the percentage identity between the sequences.

This invention provides a modified microorganism, wherein the modified microorganism comprises a modified dtxR/DtxR homologue. The invention may also provide modified microorganisms of the Streptococcus genus, wherein the modified microorganism of the Streptococcus genus comprises a modified dtxR/DtxR homologue. In these embodiments, the modified dtxR/DtxR homologue may exhibit a degree of homology/identity (as defined above) to the sequences disclosed as SEQ ID NOS: 1 and 2 herein.

Insofar as this specification relates to modified Streptococci, examples of dtxR/DtxR homologues to be exploited (i.e. modified) for production of a modified microorganism of this invention, may include those listed in Table 1 below.

TABLE 1
dtxR homologues in Streptococcus species

| | | Metal | |
|---|---|---|---|
| Organism | Regulator | ion binding | Ref/Accession |
| S. suis | ScaR (aka SloR) | Mn²⁺ | Jakubovics et al 2000 |
| S. equi | TroR | | |
| S. pyrogenes | MtsR | Mn²⁺ | Jakubovics et al 2000 |
| S. epidermidis | SirR | Mn²⁺ | CAA67572 |
| S. pneumoniae | PsaR | | Jakubovics et al 2000 |
| S. gordonii | ScaR | | AAF25184 |
| S. mutans | SloR | | Jakubovics et al 2000 |

A modified S. suis of this invention may take the form of a scaR deficient (scaR⁻) strain, genetically modified to lack a functional scaR gene or product (i.e. a functional “ScaR” protein). A modified S. suis according to this embodiment of the invention may express factors (for example virulence factors) normally under the control of ScaR in a manner which is independent of the expression, function and/or activity of ScaR.

The sequence of the S. suis scaR gene (a dtxR homologue) is given below as SEQ ID No.: 3.

SEQ ID NO: 3: S. suis scaR
atgacaccaaacaaagaagattacctaaaatgtatttatgaactgggtca
attagaccaaaaaattaccaataaactcatcgcagagaagatggccttct
ccgcaccagccgtttccgaaatgctcaaaaaaatggtagccgaagagctc
atttctaaggatgccaaggcaggttatctcctcagtcaaactgcccttga
aatggtagccagcctctatcgcaaacaccgcttgattgaggtattcttag
ttgagcaacttggctactctccagaagaagtacatgaagaggctgagatt
ttagaacacaccgtttcagatcactttatcaatcgcctagacctgctact
ggaacagcctcaaacttgtcctcacgggggaagcattcctcaagcaggac
aaccgctcatcgaacgctaccagacacggctgtcacagctaactgagaca
gggaactaccagcttgtccgtatccatgacttctatcaactccttcagta
cttggaacaacatgaattagctgtcggtgatttactaaccgtcacagcct
tcgaccagtttgcccagaccatcaccatccagtacaaggacaaagagctc
gccgtcccaacagccatcgctcaacaattattcatcgaaaaaagcaatcg
cccagcctaa

The sequence of the S. suis ScaR protein is given below as SEQ ID No.: 4.

SEQ ID NO: 4: S. suis ScaR
MTPNKEDYLKCIYELGQLDQKITNKLIAEKMAFSAPAVSEMLKKMVAEEL
ISKDAKAGYLLSQTALEMVASLYRKHRLIEVFLVEQLGYSPEEVHEEAEI
LEHTVSDHFINRLDLLLEQPQTCPHGGSIPQAGQPLIERYQTRLSQLTET
GNYQLVRIHDFYQLLQYLEQHELAVGDLLTVTAFDQFAQTITIQYKDKEL
AVPTAIAQQLFIEKSNRPA

The function and/or activity of the scaR protein is sensitive and/or response to environmental manganese concentrations. Without wishing to be bound by theory, manganese present in the environment, combines and forms complexes with ScaR; in S. suis, this results in a conformational change which allows ScaR to bind specific sequences within, or associated with, the promoter regions of target genes—for example, genes encoding ScaR-regulated microbial (S. suis) factors. As a result of the binding between ScaR/manganese complexes and sequences (for example scaR-specific nucleic acid motifs in the vicinity of promoted sequences) associated with ScaR regulated genes (encoding S. suis factors as described herein), transcription of these genes is modulated, in some cases inhibited, suppressed or prevented. While the production of internal and/or external microbial factors may be limited in manganese-rich environments, the growth of S. suis is strong and vigorous.

In contrast, in environments where manganese is unavailable or where manganese concentrations are low, ScaR does not (or cannot) complex with manganese and remains in a confirmation that is unable to bind some target sequences. As such, in the absence of manganese, ScaR-regulated promoters are not impeded from initiating transcription. However, while microorganisms such as S. suis may be able to express certain internal and/or external factors (for example virulence determinants) in environments where metal ion (in particular manganese) availability is low, microbial growth may be poor.

The inventors have discovered that S. suis ScaR-deficient strains, such as those described herein, are able to express certain factors independently of environmental manganese levels and are thus able to be cultured in manganese rich environments so as to markedly improve growth. In this way, standard laboratory culture conditions/media may be used to produce much higher amounts/concentrations of virulence factors than would otherwise be possible through culture of wild-type S. suis (i.e. scaR⁺ strains) under equivalent conditions.

In view of the above, the present invention provides modified S. suis which, under standard laboratory conditions is capable of expressing factors normally only expressed during an infection (i.e. in vivo). It should be understood that the term “standard laboratory conditions” may include environmental conditions comprising manganese and/or containing concentrations of manganese, sufficient to form ScaR/manganese complexes and inhibit or prevent expression of the factors described herein.

Furthermore, modified S. suis as described herein, can be grown in the presence of manganese while still retaining the ability to express a number of virulence factors normally under the control of the scaR protein. This is important as the presence of manganese promotes strong growth of the modified S. suis provided by this invention. Furthermore, one of skill will appreciate that a modified S. suis strain which can be grown under conditions which promote strong/vigorous growth, may be particularly well suited to vaccine production where large amounts of microbial material are required to produce sufficient quantities of vaccine.

Thus, an embodiment of this invention produces a S. suis scaR-deficient strain, wherein the strain expresses factors normally under the control of the ScaR protein, under conditions which comprise manganese concentrations sufficient to inhibit the expression of said factors in wild-type (or un-modified strains).

It should be understood that this invention may extend to any Streptococcus species within the Streptococcus genus. For example, where the invention relates to S. equi, the modified microorganism may be a strain lacking a functional troR/TroR gene/protein or a troR/TroR-deficient strain. The invention may also provide a S. pyogenes lacking (functional) or deficient in, mtsR/MtsR; S. epidermidis lacking (functional) or deficient in, sirR/SirR; S. pneumoniae lacking (functional) or deficient in, psaR/PsaR; S. gordonii lacking (functional) or deficient in scaR/ScaR; and/or S. mutans lacking (functional) or deficient in, s/OR/SloR

One of skill will appreciate that the modified (Streptococcus) microorganisms provided by this invention, may find application as strains from which vaccines may be produced.

The modified microorganism is not a modified Corynebacterium. In a further embodiment, the microorganism is not a modified C. pseudotuberculosis.

Accordingly, a second aspect of this invention provides a modified microorganism of the invention for use in raising an immune response in an animal. Moreover, the modified microorganisms described herein may be used to create vaccines for use in treating/preventing and/or controlling disease.

The invention may further provide vaccines for use in treating, preventing and/or controlling diseases caused and/or contributed to by Streptococcus species. In one embodiment, the invention provides a Streptococcus suis scaR/ScaR-deficient strain for use in raising an immune response in an animal and/or for use as a vaccine. It should be understood that any Streptococcus deficient in a dtxR-like gene/protein (for example an S. equi troR-deficient strain) may be used in treating, preventing and/or controlling diseases caused and/or contributed to by Streptococcus species.

It should be understood that the term “animal” may encompass mammalian animals including, for example, humans, equine, or ruminant (for example bovine, ovine and caprine) species, avian species and/or fish.

Where the vaccine provided by this invention is based on modified organisms of the Streptococcus genus (for example a modified S. suis or S. equi), the vaccine may find application in the treatment, prevention and/or control of diseases and/or conditions caused or contributed to by one or more Streptococci, including, for example meningitis, septicaemia, respiratory disease and/or strangles.

One of skill will appreciate that the modified microorganism, for example a modified Streptococcus, provided by this invention, may be used as a whole-cell killed vaccine. In this embodiment, the vaccine may be prepared as a bacterin vaccine, comprising a suspension of killed modified microorganisms. In other embodiments, the vaccines may comprise portions and/or fragments of the modified Streptococcus, the portions or fragments being generated by fragmentation/fractionation procedures/protocols such as, for example, sonication, freeze-thaw, osmotic lysis and/or processes which isolate sub-cellular fractions or factors secreted by the modified microorganisms into the extracellular milieu.

One of skill will appreciate that the general strategy of preparing a (bacterin) vaccine using a microorganism modified so as increase the expression of virulence factors when cultured (for example, under standard laboratory conditions (in the case of S. suis, such conditions comprising quantities of manganese sufficient to enhance or encourage growth), is somewhat at odds with routine protocols which aim to down regulate or attenuate microbial virulence factors before a microorganism is provided as a live attenuated (not killed) vaccine.

A further aspect of the invention provides a method of making any of the vaccines described herein, said method comprising the step of culturing a modified microorganism provided by this invention and preparing a vaccine composition therefrom. Vaccine compositions according to this invention and/or prepared by methods described herein, may otherwise be known as “immunogenic compositions”—such compositions being capable of eliciting host immune responses.

A method of making a modified S. suis for use in treating, preventing and/or controlling specific diseases (such as those described herein) may comprise culturing the scaR/ScaR-deficient S. suis strain described herein, under conditions which comprise manganese or manganese concentrations which would otherwise inhibit wild-type ScaR activity or function, and preparing a vaccine composition therefrom. Other streptococcal species may comprise metalo-regulatory factors which are “sensitive” to other types of metal ion—for example iron. In such cases, methods for making vaccines comprising modified forms of these species may exploit iron concentrations which would otherwise alter wild-type activity and/or function of the metallo-regulatory protein such that expression of target genes (for example genes encoding virulence factors) is modified/altered (for example inhibited or reduced).

Vaccine compositions of this invention may comprise killed forms of any of the modified microorganisms described herein and/or fragments and/or portions derived from modified microorganisms of this invention. The vaccines of this invention may be formulated together, or in combination with one or more adjuvant(s), microbial components (for example one or more bacterium or a component thereof), viral components, parasitic components, pharmaceutically acceptable carrier(s), excipient(s) and/or diluent(s).

Vaccines may be formulated and/or prepared for parenteral, mucosal, oral and/or transdermal administration. Vaccines and/or immunogenic compositions for parenetral administration may be administered interdermally, intraperitoneally, subcutaneously, intravenously or intramuscularly.

The inventors have determined that the vaccines provided by this invention, particularly vaccines comprising the modified Streptococcus organisms described above, have a number of advantages over existing vaccines. In particular, vaccines comprising the modified Streptococcus strains of this invention, exhibit superior efficacy, as the enhanced expression of virulence factors improves immune reactions within the animal or human host and need to improve protective immunity.

Moreover, production of the vaccine is simple and requires established, defined and well understood (i.e. standard) culture conditions. Additionally, by avoiding the need to alter the culture conditions (relative to culture of, for example, a wild-type strain), vaccine production is safe, simple and rapid. Moreover, since the vaccine strain is used in a killed, whole-cell form, this further simplifies the production procedure and results in a safe vaccine which can readily be combined with other killed, whole-cell type vaccines, vaccines derived from portions and/or fragments of other microorganisms (for example toxoid vaccines) as well as other forms of medicament.

One of skill will appreciate that animal vaccines are subject to withdrawal periods—i.e. the period of time an animal (or products from an animal such as milk) cannot enter the human food chain following vaccination. The withdrawal period can hinder normal farming practises and result in lost production. It is not expected that a withdrawal period will be required with bacterin (comprising a suspension of killed wild-type or modified microorganisms) type vaccine.

Following vaccination with a whole-cell killed microorganism-derived vaccine, it is often difficult to distinguish vaccinated and infected subjects. This is particularly true where both the vaccine and wild-type strains of a particular microorganism produce antigens which may be used to detect the microorganism or diagnose an infection therewith.

As such, the modified microorganisms provided by this invention may be further adapted to permit detection in a sample. For example, the modified microorganisms may comprise a detectable marker which may be exploited in a diagnostic procedure to detect or confirm the presence of a modified microorganism of this invention. One of skill will appreciate that the presence of a detectable marker in a modified microorganism of this invention would permit the identification of hosts (human or animal) which have been vaccinated with any of the modified microorganisms described herein.

The modified microorganisms of this invention may be supplemented with one or more detectable factors. In one embodiment, the detectable factor may comprise a gene and/or protein encoding a detectable factor, wherein the gene and/or protein has been introduced to a modified microorganism described herein. Genes and/or proteins of this type may be referred to as “marker genes and/or proteins”.

By way of example, a marker gene and/or protein may be introduced or delivered to a microorganism by way of a vector (for example an expression vector) such as, for example, a viral vector or a plasmid. The introduction and/or delivery of vectors to the modified microorganisms of this invention may be achieved using standard laboratory cloning procedures including those detailed in Molecular Cloning: A laboratory Manual; Sambrook and Green, Cold Spring Harbor Laboratory Press.

One of skill will appreciate that modified microorganisms further modified to include some form of detectable marker may be identified and/or detected in samples by virtue of the detectable marker. In other words, a positive identification of the detectable marker in a sample may confirm the presence of a modified microorganism of this invention.

The detectable marker may comprise a gene and/or protein which has been modified or deleted from the genome of the modified microorganism—the gene and/or protein encoding a detectable factor. One of skill will appreciate that just as the presence of a particular marker from a sample may serve to verify the presence of a modified microorganism of this invention, the absence of a particular marker from a sample, or the presence of a modified form of a particular marker from a sample, may also serve as a means to diagnose the presence of a modified microorganism of this invention.

Modified microorganisms of this invention may be further modified so as to not comprise, produce or express at least one detectable factor. In some embodiments, the detectable factor may form the basis of a standard diagnostic test.

The detectable factor may comprise or be an immunogenic protein. Advantageously, the detectable factor is one which forms the basis of a diagnostic test.

The invention provides a modified microorganism according to this invention, which modified microorganism comprises a further modification which renders it unable to express at least one other detectable factor.

One of skill will appreciate that provided the at least one other detectable factor is a factor which can be detected by some means—for example by immunological assays (for example ELISA) or molecular detection assays (for example PCR-based assays), it is possible to use the presence or absence of such a factor from samples provided or obtained from subjects to be tested, as a means of determining whether or not that subject is infected with a wild-type form of the modified microorganism (which would be expected to express the detectable factor), or has been vaccinated with the modified strain (which would have been modified to exhibit inhibited (or ablated) expression of the detectable factor). Being able to make such a distinction is important as it prevents vaccinates being mis-diagnosed as infected subjects.

The diagnostic factor may be a factor used to detect instances of infection and/or disease, caused and/or contributed to by wild-type strains of the modified microorganisms. Advantageously the diagnostic factor is an antigenic and/or immunogenic factor, and in some embodiments, the diagnostic factor may be a secreted factor.

The modified microorganisms provided by this invention may further comprise one or more detectable marker or reporter elements. The presence of such elements may further serve to distinguish vaccine strains from wild-type strains. Markers and/or reporter elements which are useful in this invention may include, for example, optically-detectable markers such as fluorescent proteins and the like.

One of skill will appreciate that while this invention relates to modified forms of Streptococci microorganisms, the teachings may be applied to other species (including species from other genera). For example, the term “modified microorganisms as used herein) may encompass modified Mycobacteria, for example modified M. tuberculosis, wherein the modified M. tuberculosis comprises a modified ideR gene and/or IdeR protein—the ideR gene and/or IdeR protein being a dtxR/DtxR homologue.

### DETAILED DESCRIPTION

The present invention will now be described in detail with reference to the following Figures which show:

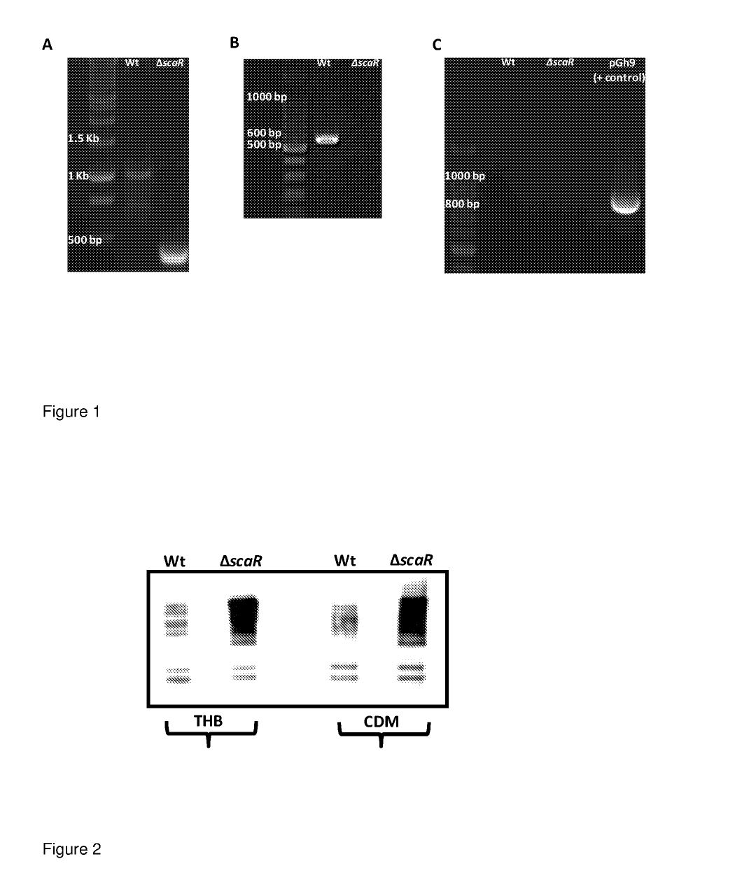

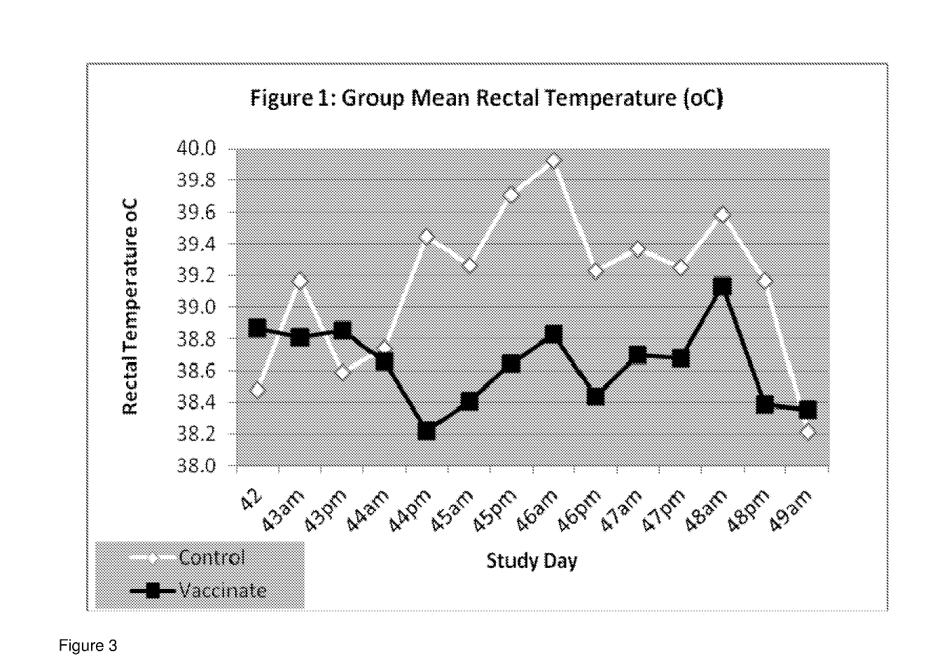

FIG. 1. PCR verification of a Streptococcus suis ΔscaR mutant. Panel A: PCR using primers flanking the deleted portion of scaR allowed amplification of an expected full-sized gene fragment (1,066 bp) from the wild-type parent strain and a shorter fragment (654 bp) from the ΔscaR mutant strain, confirming the deletion was correct and of the expected size. Lanes are annotated as shown. Panel B: PCR using primers specific for an internal portion of scaR allowed amplification of the expected sized fragment (561 bp) from the wild-type parent strain and confirmed the absence of the equivalent sequence in the ΔscaR mutant strain. Lanes are annotated as shown. Panel C: PCR analysis of the pG⁺host9-encoded erythromycin resistance gene (˜800 bp) confirmed the absence of plasmid sequences from the ΔscaR mutant strain. Lanes are annotated as shown.

FIG. 2. Western blot analysis of secreted proteins in a wild-type Streptococcus suis and isogenic ΔscaR deletion mutant. Strains were cultured in either THB or CDM before culture supernatants were TCA precipitated, separated by SDS-PAGE and then transferred onto Hybond ECL nitrocellulose membranes (Amersham Biosciences). Primary antibody (polyclonal IgG antibodies derived from convalescent pig serum following S. suis infection) was diluted 1:500 and rabbit anti-porcine IgG HRP conjugated secondary antibody (Sigma-Aldrich) was diluted 1:10,000. Immunodominant proteins were detected by ECL (Amersham-Biosciences) and images were captured using ImageQuant LAS4000 (GE Healthcare).

FIG. 3: Shows the mean rectal temperature data over the study period. The control animals were injected with sterile phosphate buffered saline at Day 0 and 28, and the vaccinated group were injected with an adjuvanted bacterin vaccine derived from a scaR mutant of S. suis at the same times. All animals were challenged with a wild type S. suis strain on Day 42.

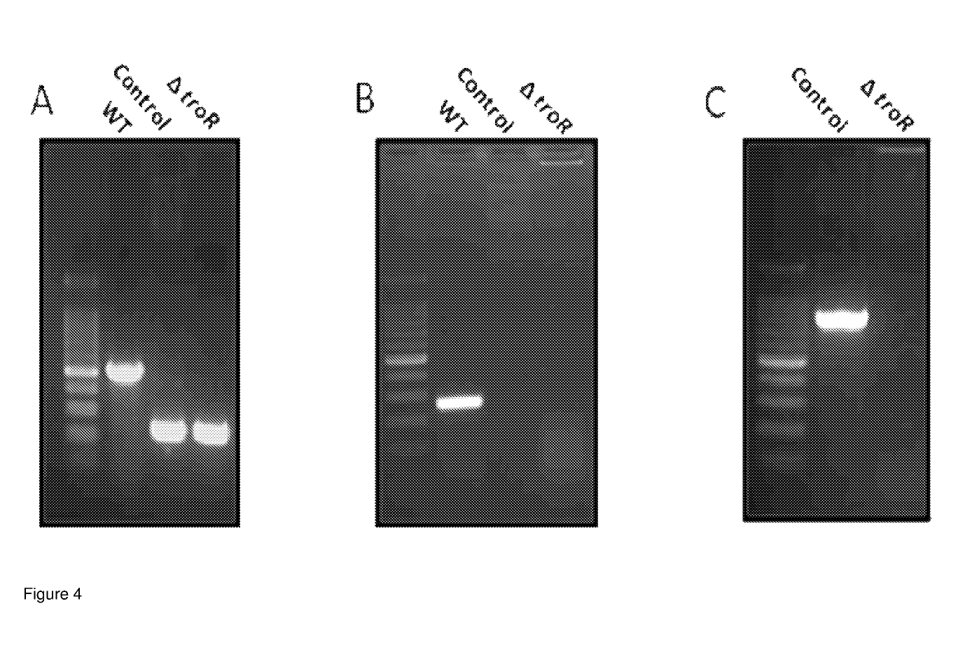

FIG. 4. PCR analysis of the Streptococcus equi ΔtroR mutant strain.

Panel A: PCR with the primers ΔtroR_ext_fwd and ΔtroR_ext_rev, which flanked the deleted portion of troR, allowed amplification of an expected full-sized gene fragment (519 bp) from the wild-type parent strain (WT) and a shorter fragment (220 bp) from the ΔtroR mutant strain (ΔtroR), confirming that the mutation was correct and of the expected size. The recombinant plasmid pGh9-ΔtroR (Control) was included as a positive control. Panel B: PCR with the primers ΔtroR_int_fwd+ΔtroR_int_rev, specific for an internal portion of troR, allowed amplification of the expected sized fragment (253 bp) from the wild-type parent strain and confirmed the absence of the equivalent sequence in the mutant strain (ΔtroR). The recombinant plasmid pGh9-ΔtroR (Control) was included as a negative control. Panel C: PCR with the primers pGh9_erm_fwd+pGh9_erm_rev, specific for the pG⁺host 9-encoded erythromycin resistance gene (erm; ca. 0.8 kb) confirmed the absence of this gene, and hence plasmid sequences from the mutant strain (ΔtroR). The recombinant plasmid pGh9-ΔtroR (Control) was included as a positive control.

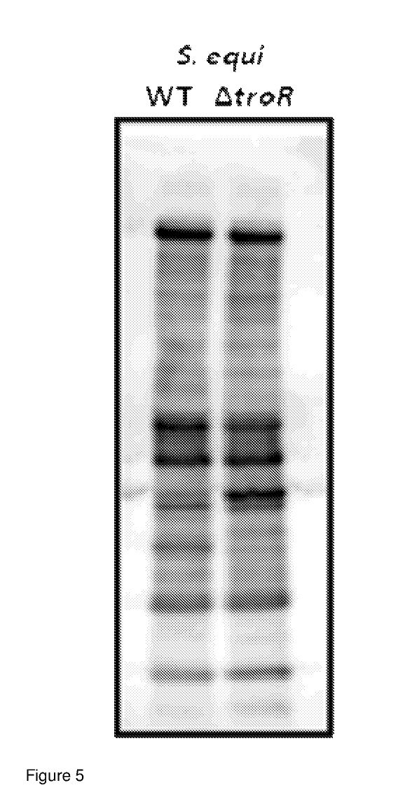

FIG. 5. Western blot analysis of secreted proteins in a wild-type Streptococcus equi and isogenic troR deletion mutant (ΔtroR). Strains were cultured in VPB before culture supernatants were precipitated, separated by SDS-PAGE and then transferred onto Hybond ECL nitrocellulose membranes (Amersham Biosciences). Primary antibody (polyclonal IgG antibodies derived from convalescent horse serum following S. equi infection) was diluted 1:500 and rabbit anti-horse IgG HRP conjugated secondary antibody (Sigma-Aldrich) was diluted 1:10,000. Immune-reactive proteins were detected by ECL (Amersham-Biosciences) and images were captured using an ImageQuant LAS4000 (GE Healthcare).

### Example 1

### Material, Methods and Results

### General Molecular Biological Techniques and Targeted Allele-Replacement Mutagenesis

Routine molecular biological manipulations were conducted as described (Sambrook et al., 1989). Transformation of E. coli and Streptococcus suis with plasmid DNA was conducted using standard procedures (Fontaine et al., 2004; Sambrook et al., 1989). Oligonucleotide primers used for PCR are described in Table 2.

Construction of a scaR (dtxR-Like Transcriptional Regulator) Mutant in Streptococcus suis

A defined scaR mutant was constructed in Streptococcus suis type strain 9682 (DSMZ). In brief, 5′ (DNA fragment A comprising 559 bp of upstream flanking sequence up to and including the translational ATG start codon of scaR) and 3′ (DNA fragment B comprising 506 bp of downstream flanking sequence encompassing the translational TAA stop codon of scaR and subsequent downstream sequence) chromosomal regions flanking the scaR gene were amplified by PCR with Phusion polymersase (Fiinzyme) in accordance with the manufacturer's guidelines using the primers detailed in Table 2. A 12 bp complementary nucleotide overlap sequence was engineered into the internal reverse primer of fragment A (Table 2) and internal forward primer of fragment B (Table 2) to increase the specificity and efficiency of the final spliced PCR reaction. The resultant amplicons (fragments A+B) were then used as a DNA template in a third cross-over PCR reaction, and the resulting DNA fragment (Fragment C) was cloned into the temperature-sensitive allele-replacement plasmid, pG⁺host 9, by virtue of primer-encoded EcoRI restriction endonuclease recognition sites. The resulting construct was designated pGh9-ΔscaR. The wild-type Streptococcus suis strain was subsequently transformed with pGh9-ΔscaR and allele replacement was conducted in an equivalent manner to that described (Fontaine et al., 2003). Following the two-step mutagenesis procedure, bacteria were plated onto solid media and potential scaR mutants were screened and verified by PCR using the primers detailed in Table 2. As expected, these primers resulted in the amplification of a ca. 1006 bp fragment from the wild-type strain; however, the equivalent PCR product for the ΔscaR strain was ca. 654 bp shorter, confirming deletion of the chromosomal scaR gene (FIG. 1, panel A). Further verification using internal scaR primers confirmed that the scaR gene was absent in the mutant strain (FIG. 1, panel B). An additional verification PCR to test for the presence of the plasmid derived erythromycin resistance gene confirmed there was no plasmid present in the scaR deletion mutant (FIG. 1, panel C). Finally, the region spanning the deleted scaR gene was PCR amplified and confirmed by sequencing (data not shown).

TABLE 2
PCR mutagenesis and verification primers

| | | |
|---|---|---|
| Primer purpose* | Sequence (5′-3′)^{†} | Reference |

Amplification of scaR flanking regions

| | | |
|---|---|---|
| scaR upstream flank F | GGGAATTCGCTACAGCTACAGCTGACTTG | This study |
| scaR upstream flank R | CGCTCAGCTTGTTTACATGAGAACTCGCTTT | |
| | C | |
| scaR downstream flank F | GAAAGCGAGTTCTCATGTAAACAAGCTGAGC | This study |
| scaR downstream flank R | G | |
| | GGGAATTCGACGAATGACGGATACTATC | |

Screening and verification of scaR mutagenesis construct and deletion mutant

| | | |
|---|---|---|
| pGh⁺9 MCS screen F | CCAGTGAGCGCGCGTAATACG | This study |
| pGh⁺9 MCS screen F | GGTATACTACTGACAGCTTCC | |
| scaR external screen F | CACAGCCACTCTTGGC | This study |
| scaR external screen R | GTCTTGCAGCCTTTAACC | |
| scaR internal screen F | GAACTGGGTCAATTAGACC | This study |
| scaR internal screen R | GAGCTCTTTGTCCTTGTAC | |
| pGh9⁺ erm screen F | TGGAAATAAGACTTAGAAGC | This study |
| pGh9⁺ erm screen R | CGACTCATAGAATTATTTCC | |

*Forward primers are denoted F and reverse primers are denoted R
^{†}Underlined sequences denote EcoRI restriction sites

Immunological Detection of S suis Secreted Proteins Using Porcine Convalescent Anti-S. suis Antibodies

In order to determine whether the abrogation of production of ScaR in the Streptococcus suis ΔscaR mutant affected the production, in vitro, of proteins normally produced in vivo during infection, a Western blot was performed using serum from a piglet challenged with Streptococcus suis. Both the scaRScaR mutant and wild-type parent strains were cultured in Todd-Hewitt Broth+1% (w/v) yeast extract (THB) or in a chemically-defined medium (CDM; Walker et al., 2011). Once mid-logarithmic growth-phase was reached, culture volumes were adjusted by measurement of absorbance at 600 nm, so that equivalent cell numbers were recovered for wild-type and mutant strains. Subsequently, cells were harvested by centrifugation and supernatant proteins were retained for further analysis. A known quantity of bovine serum albumin (BSA) was added in equivalent amounts to wild-type and mutant culture supernatants, which were then TCA-precipitated and dissolved in 1.5 M Tris-HCl (pH 7.5); the BSA subsequently served as an internal control to confirm equivalent recovery of proteins from wild-type and mutant supernatants following TCA precipitation. Equivalent volumes of wild-type and mutant-derived supernatant proteins were separated by electrophoresis through a 12% SDS-polyacrylamide gel and visualised by staining with Coomassie; equivalent amounts of BSA were observed between samples, however, several differences were observed between the secreted protein profiles of both strains (data not shown). These differences were further investigated by Western blot using polyclonal IgG antibodies derived from convalescent pig serum following S. suis infection. Results confirmed that the expression of numerous proteins was greater in the ΔscaR mutant as compared to the wild-type parent strain (FIG. 2), and equivalent results were observed for both THB and CDM-cultured bacteria. It was therefore concluded that the abrogation of production of the DtxR-like protein, ScaR, in Streptococcus suis resulted in the de-repression of some genes which are normally repressed during culture in artificial laboratory media.

### Example 2

### 9.1 Summary of Study Design

A total of eighteen piglets of 4 weeks of age were sourced from a high health status farm and housed as two groups of nine. At approximately 4 weeks of age, a blood sample was collected from each animal then one group was administered phosphate buffered saline and the other administered a formalin killed suspension of the scaR-deficient S. suis strain adjuvanted with aluminum hydroxide by intramuscular injection. These procedures were repeated four weeks later on Day 28. On Day 42, two weeks post-booster vaccination, a blood sample was collected from each animal then they were administered 5 ml of 1% acetic acid by intranasal delivery followed 1 hour later by a 5 ml volume of the challenge material by intranasal delivery at a concentration of 2×10⁸ cfu/ml. A clinical observation was carried out on the animals prior to challenge then as a minimum twice daily, for seven days. On Day 49 (or earlier if animals were euthanased early on welfare grounds) the animals were euthanased and a blood sample was collected. At necropsy samples of the brain and tonsils were removed for bacteriological assessment to determine whether the challenge isolate was present. A summary of the study design can be seen in Table 3.

TABLE 3
Summary of Treatment Groups

| | | | Dosage/ | Regime | Challenge | | Concentration | End of |
|---|---|---|---|---|---|---|---|---|
| Group | No. | Treatment | Route | (Days) | (Day 42) | Volume | (cfu/ml) | Study |
| 1 | 9 | Phosphate | 1 ml/IM | 0 + 28 | Streptococcus | 5 ml | 1.55 × 10⁸ | Day 49 |
| | | buffered | | | suis, | | | |
| | | saline | | | Serotype 2 | | | |
| 2 | 9 | Vaccine | 1 ml/IM | 0 + 28 | Streptococcus | 5 ml | 1.55 × 10⁸ | Day 49 |
| | | | | | suis, | | | |
| | | | | | Serotype 2 | | | |

IM = Intramuscular

### Test Material

Name: Streptococcus vaccine*
Dose Regime: 1 ml on two occasions (Day 0 and 28), 4 weeks apart
   *A defined scaR mutant constructed using Streptococcus suis type strain 9682 that has been formalin killed and adjuvanted with alhydrogel

### Control

Name: Sterile Phosphate Buffered Saline (PBS)
Dose Regime: 1 ml on two occasions (Day 0 and 28), 4 weeks apart

### Challenge Material

Name: Streptococcus suis, Serotype 2
Method of Administration: Intra-nasal
Anticipated Titre: 2×10⁸ colony forming units (cfu) total in 5 ml
Dose Regime: 5 ml on single occasion (Day 42)

### Test Material Administration

On Day 0, the animals from Group 1 were administered 1 ml of the control material by intramuscular injection to the right neck. All animals from Group 2 were administered 1 ml of the vaccine by intramuscular injection to the right neck. On Day 28, the animals from Group 1 were administered 1 ml of the control material by intramuscular injection to the left neck. All animals from Group 2 were administered 1 ml of the vaccine by intramuscular injection to the left neck. A new needle and syringe was used for each animal.

### Challenge Preparation

On Day 41, a microbank seed stock cryovial containing the challenge isolate was removed from −70° C. storage and placed in a pre-chilled (−70° C.±10° C.) cryoblock which was transported directly to a Microbiological Class 2 hood. Two beads were removed from the vial and streaked onto separate 5% Sheep Blood agar plates. The plates were incubated overnight for 23 hours at 37° C. Following incubation, plates were examined and confirmed as having growth consistent with that expected for the isolate. Colonies were removed from each plate and added to 4×3 ml of pre-warmed vegetable peptone broth (VPB) in bijou bottles to a turbidity of 1.5 McFarland turbidity units (McF) (density measured using a Densitometer, BioMerieux). Each 3 ml volume was added to 97 ml of pre-warmed VPB. The cultures were incubated for four hours at 37° C. on an orbital shaker set at 150 rpm. After incubation the turbidity of each culture was recorded (target was between 2.5 and 3.5 McF). 80 ml of one culture broth was removed and added to 120 ml of VPB to produce challenge material with a concentration of approximately 2×10⁸ cfu/ml (1×10⁹ cfu total in 5 ml). The challenge material was stored chilled prior to use (+2 to +8° C.). A sample of the pre and post challenge material (pooled challenge broth pre and post challenge) was used for the measurement of bacterial concentration.

### Clinical Observations

On Day 42, clinical observations were conducted prior to challenge then as a minimum twice daily from Day 43 until the end of the study. Additional observations were conducted as necessitated by the condition of the animals. Clinical observations consisted of assessments of demeanour, behavioural/central nervous system changes and rectal temperature (° C.) according to a scoring system (see Table 4). Additional comments relating to behavioural or neurological issues were recorded as comments.

TABLE 4
Clinical Observations

| | Score | | | |
|---|---|---|---|---|
| Parameter | 0 | 1 | 2 | 3 |
| Rectal Temp | 38.0° C.-39.5° C. | >39.5° C.-40.0° C. | >40.0° C.-40.9° C. | ≧41.0° C. or |
| | | | | <38° C. |
| Demeanour | Normal | Mild | Moderate | Severe |
| | | Depression | Depression | Depression |
| Description | Normal | A bit dull but | Unwilling but | Unable to rise |
| | Demeanour | active and | able to rise, | |
| | | mobile | staying apart | |
| | | | from others | |
| Behavioural/CNS | Normal | Minor Changes | Moderate | Severe |
| | | | Changes | Changes |
| Description | Normal | Lameness, | Unsteady when | Paralysis, |
| | Demeanour | tremors | walking, | involuntary |
| | | | uncoordinated, | muscle |
| | | | walking on front | movement |
| | | | knees | |

Pigs which were recumbent/moribund and/or showing signs of severe distress were euthanased immediately on humane grounds by intravenous/intraperitoneal administration of a lethal dose of Pentobarbitone Sodium BP, using a suitably sized sterile syringe and sterile needle.

### Necropsy

On Day 49 (or as required following early euthanasia on welfare grounds), animals were euthanased by lethal injection. A gross pathological examination of each carcass was conducted. Samples were collected as detailed below (see “Tissue samples”.

### Tissue Samples

At necropsy, tissue and brain samples were removed from each animal. Two samples were removed for each tissue type. One was placed in a container along with 10% formal saline for histopathological analysis, the second was placed in a sterile container for bacteriological assessment. All samples were removed using sterile forceps and scalpels to reduce risk of contamination between animals. The samples for bacteriological assessment were transported to the laboratory where they were processed on the day of collection as detailed below. The samples in formol saline were stored at ambient temperature prior to examination as detailed below under “Histological analysis”.

S. suis Culture from Tissue Samples

Each tissue sample was weighed, placed in a separate stomacher bag together with 9.0 ml of peptone water to provide a nominal dilution of 10⁻¹ and homogenised for 30 seconds in a Seward “Stomacher 80” set at high speed. The homogenate was poured into a sterile Universal Bottle labeled the 10⁻¹ dilution. A 20 μl aliquot of homogenate was diluted in 180 μl of peptone water in a sterile U-well micro titration plate to give a 10⁻² dilution. This dilution process was repeated until the homogenate was diluted to 10⁻⁷. Duplicate 10 μl aliquots of each homogenate dilution from 10⁻¹ to 10⁻⁷ were placed on the surface of a well dried 5% sheep blood agar plate. After samples are dry the plates were incubated overnight (20 to 24 hours) at 37° C. (±2° C.). Plates were inspected for typical colonies of S. suis. If present, colonies were counted.

### Histopathological Analysis

A total of ten sets of tissues (three from early deaths, four from controls and three from vaccinates were processed and examined following standard procedures

TABLE 5
Summary of study schedule
Table 5: Study Schedule

| Study Day | Procedure |
|---|---|
| Day 0 (Pre-Treatment) | Arrival, Blood Sample. Vet inspection |
| Day 0 | Administration of Vaccine/Saline |
| Day 28 | Blood Sample, Administration of |
| | Vaccine/Saline |
| Day 42 | Blood Sample, Clinical Observations, Pre- |
| | Challenge Primer (Acetic acid) |
| Day 42 (+1 hour) | Challenge |
| Day 42 - 49 | Clinical Observations |
| Day 49 | Necropsy |

### Results

### Rectal Temperature Data:

The rectal temperature data is summarised in FIG. 3. There is a considerable difference between the mean rectal temperatures when the results for the controls and vaccinates are compared. During the period between Day 44 pm and Day 46 pm the difference between the groups is around 1° C. This period (between 2 and 4 days post challenge) is the peak period for infection and this is shown by the differences between the groups. A total of 27 individual observations of rectal temperatures in excess of 39.5° C. were recorded for the control animals compared to none for the vaccinates. On Day 46 am all of the control animals had temperatures in excess of 39.5° C.

### Behaviour and Demeanour:

Only three animals (all from the control group) were recorded to have abnormal behaviour and demeanour during the study and all three animals were subsequently euthanased on welfare grounds. No vaccinate animals were observed to have any abnormal signs at any point during the monitoring period. On Day 45 (pm) Animal no. 0252 was observed to have tremors, was unsteady on its feet and appeared to be having fits, combined with a temperature of 39.7° C. On Day 46 at the morning clinicals, Animal no. 0254 was observed to be showing early signs of the disease with some lameness and minor tremors as well as a slightly depressed demeanour and a temperature of 40.4° C. Approximately 4 hours later, the animal had a temperature of 40.8° C., as well as a hunched appearance, tremors, unsteadiness and some seizures. The animal was euthanased on welfare grounds. On Day 47, Animal no. 0251 was observed to have a temperature of 40.4° C., was unable to rise, was fitting and was euthanased on welfare grounds.

### Mortality:

The mortality rate in the vaccinate group was 0% (0 out of 9) compared to 33.3% (3 out of 9) in the control group.

### Summary of Clinical Scoring:

No observations of any clinical symptoms were recorded at any stage in the vaccinate group. Only three of the control animals developed clinical symptoms following challenge, all of which were euthanased on welfare grounds. The remaining six animals in the control group all had rectal temperatures in excess of 39.5° C. on at least one occasion post challenge, suggesting that the bacteria was active within the animals, perhaps indicating a sub clinical infection, however none of these animals went on to develop clinical disease within the experimental timeframe.

### Bacteriology

A summary of the bacterial findings is shown in Table 6.

TABLE 6
Bacterial recovery from tissue samples

| | | Brain Sample | Tonsil Sample |
|---|---|---|---|
| Animal No. | Group No. | (cfu/ml) | (cfu/ml) |
| 0251 | 1 | 1.29 × 10⁴ | 4.48 × 10⁶ |
| 0252 | 1 | 1.67 × 10⁶ | 2.92 × 10⁶ |
| 0254 | 1 | 1.02 × 10⁴ | 1.52 × 10⁷ |
| 0256 | 1 | 0 | 2.29 × 10⁵ |
| 0253 | 1 | 1.06 × 10³ | 0 |

Streptococcus suis was recovered from both of the tissue samples collected from the three control animals that were euthanased prior to Day 49. A further 2 animals from the control group were also observed to have bacteria present in one tissue. The challenge bacteria could not be confirmed as present in any of the samples from the vaccinate group. The tonsil samples for the majority of the animals were heavily contaminated with other bacteria to relatively high levels and it is therefore not possible to confirm whether any of the challenge bacteria was present at lower levels. The brain samples were however clean with few if any, other bacteria present and these samples at least can be confirmed as S. suis free.

It is apparent from the data that in order for a full clinical disease to occur, sufficient numbers of S. suis must be present in the brain.

### Histopathology

A total of 10 sets of samples (brain and tonsil samples from each animal) were examined. These samples consisted of 3 animals from the vaccinated group and 7 animals from the control group (three animals which were euthanased early and four animals which were euthanased at the end of the study, but had shown no signs of clinical disease other than a transient rectal temperature increase). The results of the examination are provided in Appendix 4a and 4b and are summarised below. The three animals from the control group that were euthanased on welfare grounds prior to the end of the study were all observed to have severe active sub acute or chronic active generalised meningitis with extension into the brain along with severe chronic active necro-superative tonsillitis. These signs are consistent with infection with Streptococcus suis. Of the remaining four control animals, two were observed to have a single small focus of lymphocytes present in the brain although this was not considered to be significant, the other two along with the three vaccinate animals had no significant lesions present in the brain. The tonsil samples for these seven animals (four controls and three vaccinates) were all active with large secondary follicles and tonsilar crypts containing necrotic material, macrophages and polymorphonuclear neutrophils with colonies of small bacterial cocci. In all cases however there was no evidence of infection in the brain and the tonsilar lesions were considered to be normal for conventionally raised pigs.

### Discussion

The objective of the study was to determine whether the Streptococcus vaccine was efficacious in the control of an artificial Streptococcus suis challenge in pigs of approximately 10 weeks of age. The results of the study provide indications that the vaccine has efficacy in the prevention of the disease. No animals from the vaccinated group were observed to show any signs of clinical or sub-clinical disease during the study and all rectal temperatures stayed below 39.5° C. (considered to be the cut off for normality in pigs of this age) and no bacteria could be recovered from the tissue samples collected at post mortem. In comparison all of the control animals were recorded to have increased rectal temperatures during the study (indicative of infections or sub-clinical disease) on at least one occasion and three of them developed an acute clinical Streptococcus suis infection and were subsequently euthanased. The mortality in the control group was 33.3% and while this is not as high as had been anticipated (potentially due to animals of this age being better able to fight off the infection than younger animals), the results are still comprehensive.

The results show that the vaccine offered some protection against the challenge.

### Example 3—Streptococcus equi

### Materials & Methods

### Molecular Biological Techniques.

Routine molecular biological manipulations were conducted as described (Sambrook et al., 1989). Transformation of Escherichia coli and Streptococcus equi with plasmid DNA was conducted using standard procedures (Sambrook et al., 1989; Fontaine et al., 2004). Oligonucleotide primers used for PCR are described in Table 7.

TABLE 7
PCR mutagenesis and verification primers

| | | |
|---|---|---|
| Primer name | Description/purpose | Sequence (5′-3′)^{†} |

Amplification of troR flanking regions

| | | |
|---|---|---|
| 5′-ΔtroR_fwd | Amplification of 5′- | CGGAATTCCTTTCACCTTCTAGGTAAATCACATCAATACC |
| 5′-ΔtroR_rev | troR and upstream | GCACCCTGCGGTCTTATCCTTTACAATCCAGCCTTGTGC |
| | flanking sequence | |
| 3′-ΔtroR_fwd | Amplification of 3′- | GATAAGACCGCAGGGTGCATGATCACTTTGAGCTTATCC |
| 3′-ΔtroR_rev | troR and downstream | CGGAATTCGTGATGTTGTTGTTGCTGATCGCTTGGTGTATC |
| | flanking sequence | |

Screening and verification of troR mutagenesis construct and deletion mutant

| | | |
|---|---|---|
| ΔtroR_ext_fwd | Amplification of troR | GCAGAGAGAATGAAGGTTTCTGCAC |
| ΔtroR_ext_rev | fragment for mutant | CAATTCCTTATCTGCATAAGTGATGG |
| | screening. Primers | |
| | anneal within region | |
| ΔtroR_int_fwd | Amplification of | CTATTATCTAACAGAGCAAGGGCAG |
| ΔtroR_int_rev | internal troR fragment | TGTTTTGTTGATTTCGATTAGTGG |
| | for mutant screening | |
| pGh9_erm_fwd | Amplification of | TGGAAATAAGACTTAGAAGC |
| pGh9_erm_rev | pG⁺host 9 erm gene | CGACTCATAGAATTATTTCC |

^{†}Underlined sequences denote EcoRI restriction sites
^{‡}Multiple Cloning Site (MCS)

Construction of a troR Mutant of Streptococcus equi.

A defined troR mutant (a partial, 358 bp, in-frame deletion of the troR gene, designated ΔtroR) was constructed in Streptococcus equi subspecies equi strain 4047 (obtained from the Leibniz Institute DSMZ-German Collection of Microorganisms and Cell Cultures). Briefly, two DNA fragments were amplified from the S. equi chromosome by PCR using the primers 5′-ΔtroR_fwd+5′-ΔtroR_rev (Fragment A) and 3′-ΔtroR_fwd+3′-ΔtroR_rev (Fragment B); Fragment A comprised 708 bp of S. equi troR upstream flanking sequence, including the first 139 nucleotides of troR, while Fragment B comprised 680 bp of troR downstream flanking sequence, including the last 182 bp of troR (nucleotide positions 467-648 bp). An 18 bp complementary nucleotide overlap sequence was engineered into 5′-ΔtroR_rev and 3′-ΔtroR_fwd to increase the specificity and efficiency of a subsequent spliced PCR reaction. The resulting amplicons (Fragments A+B) were then used as DNA template in a third PCR using primers 5′-ΔtroR_fw+3′-ΔtroR_rev, and the resulting DNA fragment (Fragment C) was cloned into the temperature-sensitive allele-replacement plasmid, pG⁺host 9, by virtue of primer-encoded EcoRI restriction endonuclease recognition sites, to create the recombinant plasmid pGh9-ΔtroR. The wild-type Streptococcus equi strain 4047 was transformed with pGh9-ΔtroR and allele-replacement mutagenesis was conducted as described previously (Fontaine et al., 2003). Following the mutagenesis procedure, bacteria were plated onto solid growth media and potential troR mutants were screened by PCR to identify the desired mutant. PCR with the primers ΔtroR_ext_fwd+ΔtroR_ext_rev, which flank troR, were used to confirm the presence of a deletion within the S. equi troR gene, as was evidenced by the amplification of a ca. 0.5 kb fragment from the wild-type strain and a ca. 0.2 kb fragment from the mutant strain (FIG. 4, Panel A). In addition, PCR with the primers ΔtroR_int_fwd+ΔtroR_int_rev, which amplify a ca. 0.25 kb region of troR which is absent within the deletion derivative, confirmed the absence of this region in the mutant strain (FIG. 4, Panel B). Finally, PCR using the primers pGh9_erm_fwd+pGh9_erm_rev, which amplify a portion of the erythromycin resistance determinant (erm) of pG⁺host 9, failed to detect this sequence confirming that the plasmid had been lost from the chromosome (FIG. 4, Panel C). The region spanning the deleted troR gene was then amplified by PCR and sequenced to confirm that the mutation was as expected (data not shown).

Immunological Detection of S. equi Secreted Proteins by Convalescent Serum from a Horse with Strangles.

In order to determine whether the abrogation of production of TroR in the Streptococcus equi ΔtroR mutant affected the production, in vitro, of proteins normally produced in vivo during infection, a Western blot was performed using serum from a horse that had recovered from strangles infection. Both the troR mutant and wild-type parent strain were cultured in TSE compliant Veggitone Vegetable Peptone Broth (VPB). Once mid-logarithmic growth-phase was reached, culture volumes were adjusted by measurement of absorbance at 600 nm, so that equivalent cell numbers were recovered for wild-type and mutant strains.

Subsequently, cells were harvested by centrifugation and supernatant proteins were retained for further analysis. A known quantity of bovine serum albumin (BSA) was added in equivalent amounts to wild-type and mutant culture supernatants, which were then TCA-precipitated and dissolved in 1.5 M Tris-HCl (pH 7.5); the BSA subsequently served as an internal control to confirm equivalent recovery of proteins from wild-type and mutant supernatants following TCA precipitation. Equivalent volumes of wild-type and mutant-derived supernatant proteins were separated by electrophoresis through a 12% SDS-polyacrylamide gel and visualised by staining with Coomassie Brilliant Blue stain; equivalent amounts of BSA were observed between samples; however, several differences were observed between the secreted protein profiles of both strains (data not shown). These differences were further investigated by Western blot using polyclonal IgG antibodies derived from convalescent equine serum following natural S. equi infection. Results confirmed that the expression of some proteins was greater in the ΔtroR mutant as compared to the wild-type parent strain (FIG. 5) implying de-repression of target genes as a result of the genetic disruption of troR.

### REFERENCES

Fontaine, M C., Lee J J and Kehoe M (2003). Combined contributions of streptolysin O and streptolysin S to virulence of serotype M5 Streptococcus pyogenes strain Manfredo. Infect Immun 71(7): 3857-3865.
Fontaine M C, Perez-Casal J, Willson P J (2004). Investigation of a novel DNase of Streptococcus suis serotype 2. Infect Immun 72(2):774-81.
Sambrook, J., Fritsch, E. F. & Maniatis, T. (1989) Molecular Cloning: A laboratory manual, 2nd Ed., Cold Spring Harbor Laboratory Press, Cold Spring Harbor, N.Y., pp 1.63-1.70.
Walker C A, Donachie W, Smith D G, Fontaine M C. (2011). Targeted allele replacement mutagenesis of Corynebacterium pseudotuberculosis. Appl Environ Microbiol 77(10): 3532-3535.
Sambrook, J. and Russell, D. W. 2001. Molecular cloning: a laboratory manual, 3rd ed., Cold Spring Harbor Laboratory Press, Cold Spring Harbor, N.Y.
Fontaine, M. C., Perez-Casal, J. and Willson, P. J. 2004. Investigation of a novel DNase of Streptococcus suis serotype 2. Infect Immun 72(2):774-81.

1. A modified microorganism capable of expressing at least one factor under conditions in which a wild-type or unmodified strain of the same microorganism, exhibits inhibited expression of the at least one factor.

2. The modified microorganism of claim 1, wherein the modified microorganism is a Streptococcus species.

3. The modified microorganism of claim 2, wherein the Streptococcus species is selected from the group consisting of S. suis, S. equi, S. pyrogenes, S. epidermidis, S. pneumoniae, S. gordonii, S. mutans, S. uberis, S iniae, S agalactiae, S. pneumoniae, S. oralis, S. dysgalactiae and members of virdans group.

4. The modified microorganism of claim 1, wherein the modified microorganism comprises one or more genetic modification(s) which directly and/or indirectly affect the expression, activity and/or function of one or more microbial regulatory elements.

5. The modified microorganism of claim 1, wherein the one or more microbial regulatory elements comprises an environmentally sensitive or responsive control element.

6. The modified microorganism of claim 1, wherein the at least one factor is a virulence factor.

7. The modified microorganism of claim 1, wherein the modified microorganism exhibits increased expression of one or more virulence factors.

8. The modified microorganism of claim 1, wherein the modified microorganism:
(i) comprises a modified dtxR/DtxR homologue
(ii) lacks a functional DtxR homologue;
(iii) lacks a gene functionally equivalent to dtxR; and/or
(iv) lacks a gene or protein which is dtxR/DtxR like.

9. The modified microorganism of claim 8, wherein a dtxR/DtxR homologue, a gene functionally equivalent to dtxR and/or a gene or protein which is dtxR/DtxR like exhibits a degree of homology or identity to/with SEQ ID NOS: 1 or 2 or fragments thereof.

10. A modified microorganism of the Streptococcus genus comprising a modified dtxR/DtxR homologue, wherein the dtxR/DtxR homologue exhibits a degree of homology/identity to the sequences disclosed as SEQ ID NOS: 1 and 2.

11. The modified microorganism of claim 10, wherein the dtxR/DtxR homologue exhibits at least 65% homology or identity to/with SEQ ID NOS: 1 or 2

12. The modified microorganism of claim 10, wherein the microorganism is:
(i) a Streptococcus suis lacking a functional scaR gene or product; or
(ii) a scaR and/or ScaR deficient Streptococcus suis;
wherein the S. suis of (i) and (ii) expresses factors and/or virulence factors normally under the control of scaR/ScaR in a manner which is independent of the expression, function and/or activity of scaR/ScaR.

13. The modified microorganism of claim 10, wherein the microorganism is:
(i) a Streptococcus equi lacking a functional troR gene; or
(ii) a troR and/or TroR deficient Streptococcus equi;
wherein the S. equi of (i) and (ii) expresses factors and/or virulence factors normally under the control of troR/TroR in a manner which is independent of the expression, function and/or activity of troR/TroR.

14. The modified microorganism of claim 10, wherein the microorganism is selected from the group consisting of:
(i) a mtsR/MtsR-deficient S. pyogenes;
(ii) a S. pyogenes lacking a functional mtsR/MtsR gene and/or product;
(iii) a sirR/SirR-deficient S. epidermidis;
(iv) a S. epidermidis lacking a functional sirR/SirR gene and/or product;
(v) a psaR/PsaR-deficient S. pneumoniae;
(vi) a S. pneumoniae lacking a functional psaR/PsaR gene and/or product;
(vii) a scaR/ScaR-deficient S. gordonii;
(viii) a S. gordonii lacking a functional scaR/ScaR gene and/or product;
(ix) a sloR/SloR-deficient S. mutans; and
(xi) a S. mutans lacking a functional sloR/SloR gene and/or product.

15. A vaccine comprising a modified microorganism according to claim 1.

16-25. (canceled)

26. The vaccine of claim 15, wherein the vaccine comprises a killed modified microorganism and/or a killed modified Streptococcus.

27. The vaccine of claim 15, wherein the vaccine comprises one or more additional components selected from the group consisting of:
(i) an unmodified bacterium or component of;
(ii) a viral component;
(iii) a parasitic component; and
(iv) an adjuvant, a pharmaceutically acceptable carrier, excipient and/or diluent.

28. A method of making the vaccine of claim 15, said method comprising the step of culturing a modified microorganism capable of expressing at least one factor under conditions in which a wild-type or unmodified strain of the same microorganism, exhibits inhibited expression of the at least one factor and preparing a vaccine composition therefrom.

29. A vaccine comprising a modified microorganism of the Streptococcus genus comprising a modified dtxR/DtxR homologue, wherein the dtxR/DtxR homologue exhibits a degree of homology/identity to the sequences disclosed as SEQ ID NOS: 1 and 2.

30. The vaccine of claim 29, wherein the modified microorganism is killed modified.

31. The vaccine of claim 29, wherein the vaccine comprises one or more additional components selected from the group consisting of:
(i) an unmodified bacterium or component of;
(ii) a viral component;
(iii) a parasitic component; and
(iv) an adjuvant, a pharmaceutically acceptable carrier, excipient and/or diluent.

32. A method of making the vaccine of claim 29, said method comprising the step of culturing a modified microorganism of the Streptococcus genus comprising a modified dtxR/DtxR homologue, wherein the dtxR/DtxR homologue exhibits a degree of homology/identity to the sequences disclosed as SEQ ID NOS: 1 and 2, and preparing a vaccine composition therefrom.

33. A method of raising an immune response in an animal, said method comprising administering an immunogenic amount of the modified microorganism of claim 1.

34. A method of raising an immune response in an animal, said method comprising administering an immunogenic amount of the modified microorganism of claim 10.

35. The modified microorganism of claim 1, wherein the modified microorganism is adapted to permit detection in a sample.

36. The modified microorganism of claim 35, wherein the modified microorganism comprises or expresses a detectable marker, one or more detectable factors, a gene and/or protein encoding a detectable factor.

37. The modified microorganism of claim 36, wherein the gene encoding a detectable factor comprises a exogenous gene.

38. The modified microorganism of claim 10, wherein the modified microorganism is adapted to permit detection in a sample.

39. The modified microorganism of claim 38, wherein the modified microorganism comprises or expresses a detectable marker, one or more detectable factors, a gene and/or protein encoding a detectable factor.

40. The modified microorganism of claim 39, wherein the gene encoding a detectable factor comprises a exogenous gene.

41. The modified microorganism of claim 1, wherein the modified microorganism is further modified so as to not comprise, produce or express at least one detectable factor, which factor comprises an immunogenic protein and/or forms the basis of a standard diagnostic test.

42. The modified microorganism of claim 10, wherein the modified microorganism is further modified so as to not comprise, produce or express at least one detectable factor, which factor comprises an immunogenic protein and/or forms the basis of a standard diagnostic test.
